SEQUENCE LISTING (2 sequences)

SEQ ID NO: 1 - DNA, 16 bp, Artificial Sequence
  acrydite-modified oligonucleotide
tttttcacagatgagt

SEQ ID NO: 2 - DNA, 16 bp, Artificial Sequence
  acrydite-modified oligonucleotide
ttttcccaggttctct

(19) United States
(12) Patent Application Publication
(10) Pub. No.: US 2012/0228520 A1
(43) Pub. Date: Sep. 13, 2012

(54) Photoregulated Reversible Hydrogels for Delivery and Releasing of Drugs and Other Therapeutical Reagents

(76) Inventors: Weihong Tan, Gainesville, FL (US); Huaizhi Kang, Beijing (CN)

(21) Appl. No.: 13/390,754

(22) PCT Filed: Aug. 12, 2010
(86) PCT No.: PCT/US10/45314

§ 371 Date: May 11, 2012

Related U.S. Application Data
(60) Provisional application No. 61/235,040, filed on Aug. 19, 2009.

(51) Int. Cl. G21K 5/04; A61K 47/32

(52) U.S. Cl. 250/492.1; 514/772.4

(57) ABSTRACT

A novel hydrogel delivery systems useful for encapsulating and releasing pharmaceuticals or chemicals is disclosed where water soluble polymers containing crosslinker repeating units that associate or dissociate with complementary crosslinking repeating units or separate linkers to reversibly crosslink the hydrogel. In an exemplary embodiment, a DNA crosslinked hydrogel displays photoreversibility. An exemplary hydrogel delivery system comprises DNA polymer conjugates, wherein complementary DNA sequences are crosslinked with polymer chains and hybridization of the DNA sequences is controlled by photoresponsive moieties. Such hydrogels can be used to release drug molecules and/or other therapeutic reagents. The exemplary hydrogel employs photosensitive azobenzene moieties that are incorporated into the DNA crosslinker units. The azobenzene moieties respond to different wavelengths of light so that the state of azobenzene isomerization is induced by the proportion of visible and UV light irradiated. The isomer state of the azobenzene dictates whether the complementary DNA sequences hybridize to cross link the DNA polymer conjugates. Thus, irradiation of light (visible or UV) can transform the hydrogel network between a sol and any of multiple gel states to regulate the degree of crosslinking between complementary DNA sequences and, therefore, provide a profile of release of a hydrogel encapsulated pharmaceutical or other chemical.

### CROSS-REFERENCE TO A RELATED APPLICATION

This application claims the benefit of U.S. provisional application Ser. No. 61/235,040, filed Aug. 19, 2009, which is incorporated herein by reference in its entirety.

### GOVERNMENT SUPPORT

The subject invention was made with government support under National Institute of Health grant number R01 GM079359. The government has certain rights in the invention.

### BACKGROUND OF INVENTION

A hydrogel is a network of polymer chains that is water-insoluble. Hydrogels are superabsorbent and possess a degree of flexibility very similar to natural tissue. In recent years, hydrogels have been explored extensively as biomaterials in complex functional devices, tissue growth, and pharmaceutical carriers. Besides being building materials for device fabrication and biosensor development, hydrogels have been developed for response only on exposure to an external stimulus such as temperature changes, photons, ions, proteins and DNA. Hydrogels that undergo physicochemical changes upon the application of stimuli to induce chemical and physical changes of the gel are of great interest for biomedical research, drug development and clinical applications.

Currently, chemotherapy development requires not only novel drugs but also delivery systems for controllable delivery. The serious side effect or toxicity of most available drugs highly demands efficient and safe delivery methods for controllable treatment. An ideal drug delivery system should carry the drug to the location where it is required and “smartly” release it and mainly includes a multifunctional carrier for drug containing, transporting and controllable releasing. Such drug delivery systems have been intensively investigated and produce a rapidly expanding area for global therapeutic drug market. Currently there are several types of applicable models for drug delivery such as liposome and micelle, biofunctionalized nanoparticles and macromolecules (including polymers, dendrimers and hydrogels) under investigation. Among these, hydrogels are one type of promising material due to several advantages:

1. Hydrogels are composed of water soluble, three-dimension polymer materials that are crosslinked to form water swellable but insoluble networks. As noted above, they are superabsorbent and possess flexibility and high viscoelasticity very similar to natural tissue.

2. Hydrogels can be chemically modified to inert surface with resistance to outer environment, and minimize nonspecific interactions.

3. Hydrogels can be sophisticatedly tuned in morphology by adjusting each component and preparation methodologies, so that mechanical behaviors are controllable.

4. The encapsulation capability of hydrogels is based on physical entrapment and does not alter properties of loaded materials, so that they can be applied to a wide variety of pharmaceuticals, such as small molecule drug, therapeutic nanomaterials, bioactive proteins and genes.

5. Some hydrogels can undergo physicochemical gel-sol conversion in response to external stimuli such as pH, temperature, biomolecular targets, magnetic and electronic fields and photon.

While most of the materials that comprise functional hydrogels are mono-components, such as polymer chains or pure DNA, hybrid materials have recently been developed to construct multifunctional hydrogels. For example, several DNA-based nanostructures incorporated with azobenzene moiety have also been constructed and investigated with photoregulated capability. See Asanuma, H. et al. Angew. Chem. Int. Ed., 40:2671-2673 (2001); Liang, X. et al. Tetrahedron Lett., 42:6723-6725 (2001); Asanuma, H. et al. Chembiochem, 2:39-44 (2001); Liang, X. et al. J. Am. Chem. Soc., 124:1877-1883 (2002); Asanuma, H. et al. Nucleic Acids Symp. Ser., 49:35-36 (2005); Liang, X. et al. Chem Bio Chem, 9:702-705 (2008); and Takahashi, K. et al, LNCS, 3892:336-346 (2006). Azobenzene molecules have been extensively studied for their reversible isomerization between planar trans-form and non-planar cis-form under UV and visible light irradiation. See Sudesh, G.; Neckers, D. C. Chem. Rev., 89:1915-1925 (1989); Berg, R. H. et al. Nature, 383, 505-508 (1996); Guang Diau, E. W. J. Phys. Chem. A, 108:950-956 (2004); and Zhao, Y.-L. and Stoddart, J. F. Langmuir, DOI:10.1021/1a804316u. Unfortunately, a photoregulated DNA crosslinked polymeric hydrogel has not been disclosed, where change from gel to sol is photoregulated via a DNA crosslinkage.

Although the above mentioned studies have described light-induced changes in the shape of polymers and gels, none has combined DNA and DNA-polymer conjugates to create photoregulated DNA crosslinked hydrogels nor has anyone offered a satisfactory system or method of using localized irradiation to accomplish controlled, photo-actuated drug release from an implantable device while adequately avoiding potential damage to the surrounding body tissue.

### BRIEF SUMMARY

The subject invention relates to hydrogel delivery systems with photocontrollable pharmaceutical releasing capability. In a particular embodiment, the subject invention provides a new type of DNA crosslinked polymer hydrogel with reversible photocontrollability.

In principle, many physicochemical changes can be used to stimulate drug release from hydrogels where the hydrogel is converted between sol-gel states. A convenient stimulus is light, i.e. upon photon illumination, to drive the hydrogel sol-gel conversion. Using photon energy to drive hydrogel gel-sol conversion can be easily and effectively performed with photons of different wavelengths. Moreover, photon-initiated response can induce precisely localized changes in physical and chemical properties with excellent spatial resolution. Photon energy is also a clean energy source, and can be applied in otherwise unattainable environments due to the easy transportation of light through optical fiber and waveguides. In addition, photons with longer wavelength can be introduced for faster and deeper penetration through biological samples like a human body. According to the subject invention, the ability to use photons to control hydrogel release of loads has many important applications in basic research, controlled release of drugs and clinical practice.

Advantageously, embodiments of the subject invention provide highly efficient releasing systems for photocontrollable release of a pharmaceutical load at regions under light irradiation. The disclosed hydrogel systems are simple, reproducible and highly adaptable for delivering different materials, including small molecule drugs or chemicals, nanoparticles, and bioactive enzyme, with excellent localization and controllability.

In a specific embodiment of the invention, the hydrogel delivery systems comprise DNA polymer conjugates having short DNA sequences tethered to linear polyacrylamide chains that comprise the hydrogel backbone, DNA linkers to crosslink the polymer chains, and drug molecules and/or other therapeutical reagents. In this embodiment the DNA linkers comprise at least one azobenzene moiety (such as azobenzene phosphoramidite). The azobenzenc DNA linkers (ADLs) contain sequences to crosslink with a plurality of complementary DNA strands that are independently branched from the DNA polymer conjugates and form the hydrogel network. These azobenzene based hydrogels can be easily converted from gel to sol or sol to gel by photoinduced changes of the azobenzene moieties' conformation from trans- to cis- or cis- to trans- upon irradiation. These novel photoregulated hydrogels display controlled reversibility, where the sol-gel conversion can be used to encapsulate and release selected loads. This photocontrollable hybrid material advantageously permits the application of photon energy to drive reversible sol-gel conversion by cycling UV and visible irradiations where tuning of the equilibrium can be carried out by the sequence and structure of the DNA-based crosslinker units employed. The hydrogels can be modified as desired by controlling the functionality to enhance the loading and release properties of the hydrogel.

As such, methods, devices and compositions for the photo-modulated release of a chemical from a DNA-crosslinked hydrogel are provided by the present invention. In a particular embodiment, methods, devices and compositions for the in vivo localized, photo-controlled release of a therapeutic agent, such as a drug, from an implanted DNA crosslinked hydrogel are provided by the present invention. These methods, devices and compositions offer the ability to localize light irradiation and avoid potential damage to the surrounding tissue to a greater extent than is possible with existing methods and devices. The new composites, and their methods of use, are compatible with many types of therapeutic agents, including chemicals, drugs, proteins and oligonucleotides. The modulation is highly repeatable, allowing use of one device for many dosages.

One advantage of the present methods and composites is an ability to locally change the conformation of photo-responsive DNA crosslinked hydrogel material by exposure to light targeted for DNA linkers comprising at least one azobenzene moiety. The azobenzene moiety, upon exposure to light, changes conformation thus breaking the DNA crosslinkage, which permits the hydrogel and ultimately the sol to assume other conformation. This allows implantation of a drug delivery device with multiple dosages, and provides for external control over the dosage profile by regulating the device's exposure to an appropriate light source.

The subject invention further pertains to the methods for fabrication of the hydrogels of the subject invention as well as to the use of these hydrogels.

### BRIEF DESCRIPTION OF DRAWINGS

FIG. 1 shows the mechanism and design of photocontrollable DNA crosslinked hydrogels. (a) Azo-incorporated DNA linkers crosslink the DNA-polymer conjugates to form the hydrogel where the hydrogel encapsulates loads while converting from the sol state. (b) Shows the design of ADL- and DNA-polymer conjugates where two 12 base DNA segments from DNA-polymer conjugates and a 24 base ADL can complex under proper conditions to crosslink the polymer chains, resulting in a hydrogel.

FIG. 2 shows the photosensitivity of ADL and DL crosslinked hydrogels, (a) Reversible gel-sol conversion from UV-visible irradiations on a 300 μM hydrogel. The gel began to respond at 2 minutes and is effectively a sol after 20 minutes. The sol state rapidly re-gelled by visible light irradiation (top). The same hydrogel irradiated solely by visible light displayed little flow. (b) Shows the response to continuous UV light of a crosslinked hydrogel prepared with 300 μM DNA linker that is free of azobenzene moieties.

FIG. 3 shows the structure of regulated ADL hydrogel matrix. The estimated pore size is based on the ratio of acrydite-modified DNA and acrylamide, length of crosslinked DNA, and the angle of chemical bonds.

FIG. 4 shows a scheme of reversible sol-gel conversion by UV and visible light. The irregular crosslinking between random complementary DNA strands varies by actual cage size and transition conditions.

FIG. 5 shows controllable release of ADL crosslinked hydrogels loaded with different materials. (a) Model of and size/natural effect on different types of loads to hydrogel controllable encapsulation and release. (b) UV irradiation of fluorescein-encapsulated ADL hydrogel (300 μM) with initial release of dye molecules after about 2 minutes. (c) Controllable release of 500 nM 13 nm gold NPs mixed with three different concentrations of ADL hydrogels. The mixtures were each detained inside a quartz microcell with buffer solution on top and were irradiated by visible light and UV light. The absorption at 520 nm was monitored. The absorption values were normalized by setting pure buffer absorption as 0% and 100 nM NPs solution as 100% (The right top insertion is the setting of microcell for absorption measurement.). (d) Controllable release of HRP enzyme encapsulated in hydrogels quantitatively calculated by catalyzing luminol oxidation. The reaction was monitored by chemiluminescence on 410 nm. However, the timeline of this experiment is not comparable to that of the gold NPs by reason of the different measurement procedure applied. All experiments were repeated at least three times.

FIG. 6 shows thermodynamic release of gold nanoparticles (NPs) controlled by increase of temperature for hydrogels loaded with 13 nm gold NPs at an initial concentration of 500 nM. All hydrogels were irradiated by visible light for 10 minutes at 20° C. before increasing the temperature. The absorption at 520 nm was monitored every 10 degrees where 100% absorbance was set to correspond to the absorbance measured for 100 nM gold NPs in buffer solution with all experiments repeated three or more times.

FIG. 7 shows biocompatibility of human leukemia CEM cells with ADL and ADL crosslinked hydrogels. Different concentrations of ADL and ADL crosslinked hydrogels were incubated with the same amount of CEM cells (1 million/ml). CEM cells at 37° C. generally can double the population with sufficient culture media: (a) living cells under different concentrations of ADL (1, 10, 100, 300 μM), (b) living cells under different concentrations of ADL hydrogels (10, 100, 300, 1000 μM)) on CEM cells, and (c) distribution of different species of cells with different concentration of ADL (10 μL of each gel was applied to identical 90 μL cell medium, and cell proliferation was monitored on a timeline; the cell proliferation was monitored by flow cytometry and repeated at least three times).

### BRIEF DESCRIPTION OF THE SEQUENCES

SEQ ID NO. 1 is a nucleic acid sequence modified with acrydite in accordance with an embodiment of the invention.

SEQ ID NO. 2 is a nucleic acid sequence modified with acrydite in accordance with an embodiment of the invention.

### DETAILED DISCLOSURE

Embodiments of the invention are directed to photoregulated reversible hydrogel delivery systems where a load is delivered by photoinducing a change in the crosslink density of the hydrogel, including total conversion to a sol state or a maximally crosslinked gel. The hydrogel delivery system comprises a water soluble polymer comprising a multiplicity of water soluble repeating units and a plurality of crosslinker repeating units that form a plurality of crosslinks for complementary reversible association with a second crosslinker repeating unit and/or a linking molecule wherein a gel is formed upon association and a sol exists as the equilibrium approaches a fully dissociated state. The association-disassociation equilibrium is determined by the structure of the crosslinking repeating units and/or the linkers. The crosslinking repeating units and/or the linker comprises a group that can be photochemically converted reversibly from one isomeric form or another where, in one isomeric form association is facile while in another isomeric form association is inhibited. Association is promoted by irradiation at one wavelength or range of wavelengths and dissociation is promoted by irradiation at another wavelength or range of wavelengths. Typically the range will include a maximum of absorbance and herein where a wavelength is disclosed it should be understood that a range of wavelengths including the wavelength may be employed. The equilibrium populations of the crosslinked and free crosslinker repeating units are changed on demand by the irradiation of the hydrogel by light at one or more desired wavelengths or range of wavelengths where the rate at which the new equilibrium populations are established can be controlled by the ratio of intensities of irradiation at the wavelengths.

The association-dissociation occurs between crosslinker units and/or via linking units, which comprise pairs of complementary monomeric or oligomeric chains that promote specific interactions between the pairs where one or more of the crosslinker units and/or linking units contain one or more of the isomerizable groups. Simultaneous irradiation at both wavelengths can establish an equilibrium state that depends on the relative intensities of radiant energy provided at the two wavelengths. In one embodiment of the invention, one crosslinker repeating unit comprises a photoresponsive isomerizable group and a complementary crosslinker repeating unit that is free of a photoresponsive isomerizable group. In another embodiment of the invention, crosslinker repeating units of the water soluble polymer are linked to components or nucleotide sequences that are free of photoresponsive isomerizable groups and include complementary linking units (that can hybridize and/or otherwise link with the components or nucleotide sequences) that contain one or more photoresponsive isomerizable groups. In another embodiment of the invention, crosslinker repeating units of the water soluble polymer include components and/or nucleotide sequences that include photoresponsive isomerizable groups and complementary linking units (that can hybridize and/or otherwise link with the components or nucleotide sequences) that are free of photoresponsive isomerizable groups. All crosslinker repeating units and linking units can contain one or more photoresponsive isomerizable groups.

In certain embodiments, a crosslinker comprises a pair of complementary nucleic acid sequences (also referred to herein as chains or units). The complementary nucleic acid sequences are each independently about 3 to about 150 nucleotides in length, preferably about 4 to about 100 nucleotides in length, more preferably between about 5 to about 50 nucleotides in length. The nucleic acid sequences are complementary (preferably over at least about 5%, 10%, 20%, 30%, 40%, 50%, 60%, 70%, 80%, 90% or greater of their respective lengths) to each other so as to hybridize or associate with each other. Percentage complementary can be readily determined by routine experimentation or preferably by use of a suitable computer software program such as BLAST and related programs. Disclosure relating to using the BLAST program can be found at the United States National Center of Biotechnology Information (NCBI).

The nucleic acid sequences are made from DNA (deoxyribonucleic acid), RNA (ribonucleic acid), or mixtures of DNA and RNA. The nucleic acid may contain modified nucleotides including abasic nucleotides and nucleotides with additional chemical moieties linked thereto for the purpose of the invention. The links between the nucleotides may include bonds other than phosphodiester bonds, for example, peptide bonds as in PNA (peptide nucleic acid). Modified inter-nucleotide linkages are well known in the art and include methylphosphonates, phosphorothioates, phosphorodithionates, phosphoroamidites, and phosphate ester linkages. Dephospho-linkages are also known and include siloxane, carbonate, carboxylmethyl ester, acetamidate, carbamate, and thioether linkages. Plastic DNA, having for example N-vinyl, methacryloxyethyl, methacrylamide or ethyleneimine intemucleotide linkages can also be used. See e.g., Uhlmann and Peyman, “Antisense oligonucleotides: a new therapeutic principle,” Chem. Rev. 90: 543-584 (1990). PNA is particularly useful because it is resistant to degradation by nucleases and also because it forms a stronger hybridized duplex with natural nucleic acids. See e.g., Orum et al., “Single base pair mutation analysis by PNA directed PCR clamping,” Nucleic Acids Res. 21:5332-5336 (1993) and Egholm, M. et al., “PNA hybridizes to complementary oligonucleotides obeying the Watson-crick hydrogen-bonding rules,” Nature, 365:566-568 (1993). See also U.S. Pat. No. 5,925,517 for related disclosure.

In a preferred embodiment, crosslinker units comprise a pair of complementary DNA chains, wherein each of the complementary DNA chains is copolymerized with, attached to, or coupled with a water soluble polymer and wherein one of the complementary DNA chains includes a photoresponsive isomerizable group.

In alternative embodiments, the crosslinker comprises a pair of components (also referred to herein as units) that are operably linked or associated with each other. For example, the components may be operably linked or associated with each other via covalent linkage, such as an association through carbon-carbon bonding, carbon-oxygen bonding, phosphodiester bonding, peptide bonding, and the like. However, in other embodiments, the components may be operably linked or associated with each other via non-covalent linkages such as by hydrogen bonding, salt bridges, ionic attractions, and the like.

According to the subject invention, the character of the linkage, association, or hybridization of the pair of components or nucleic acid chains of a crosslinker unit is controlled by the photoresponsive isomerizable group(s). For instance, where the crosslinker unit includes a pair of operably linked or associated components, one of the components includes a photoresponsive isomerizable group. The photoresponsive isomerizable group can be photochemically and reversibly converted from one isomeric form or another where in one isomeric form the pair of components are operably linked or associated, while in another isomeric form the pair of components are disassociated or not linked with one another.

Where the crosslinker unit includes a pair of complementary nucleic acid sequences that can hybridize to each other, one of the nucleic acid sequences includes a photoresponsive isomerizable group. The photoresponsive isomerizable group can be photochemically and reversibly converted from one isomeric form to another, where in one isomeric form the pair of nucleic acid chains are hybridized, while in another isomeric form the pair of nucleic acid chains are disassociated or not hybridized with one another.

In other embodiments of the invention, the crosslinker can comprise: components or nucleic acid sequences attached to the water soluble polymer and a linking unit that includes a photoresponsive isomerizable group. The linking unit further includes a component or nucleic acid sequence that is complementary to or links with those components or nucleic acid sequences linked to the water soluble polymer.

The water soluble repeating units can be of any known water soluble polymer where the polymer is of sufficiently high molecular weight that it is not toxic to an organism to which the hydrogel delivery system is employed such that the fully dissociated sol state does not endanger the organism. Repeating units that can be used include, but are not exclusive to, those from polyacrylamide, polyvinyl alcohol, polyacrylic acid, polyethylene oxide, or modified cellulose. With exception of the crosslinker repeating units, the water soluble polymer can be a homopolymer, a copolymer of two or more water soluble repeating units or a copolymer of one or more repeating units of a water soluble homopolymer and one or more repeating units of a water insoluble homopolymer such that the copolymer is water soluble.

The repeating units can be neutral species or ionic in nature. For example the water soluble polymer can include repeating units of acrylic acid and/or repeating units of the corresponding salt form, such as sodium acrylate. The water soluble polymers can be linear, branched, hyperbranched or dendritic.

The crosslinker unit can be any unit capable of copolymerizing with, attached to, or coupled with the water soluble repeating units of the water soluble polymer. On average, at least two crosslinker repeating units are included in the water soluble polymer chain. In one embodiment of the invention the crosslinker repeating units include an oligomeric DNA chain. In addition to oligomeric DNA chains, other groups capable of forming specific complexes in aqueous solution can be employed. The complexation can involve multiple hydrogen bonding as with DNA association or can involve other polar, ionic or van der Waals interactions. In some embodiments of the invention, an equilibrium involving formation and breaking of covalent bonds can be carried out, for example, via formation or breaking of a ring via a diets alder cycloaddition reaction.

The isomerizable moiety can be one that involves a change that involves isomerization about one or more pi bonds or can involve a ring-chain interconversion. For example, in a non-limiting manner, the moiety can be an azobenzene moiety or a 1,2-diphenylethylene moiety. Phenyl rings can be mono, di or polyaromatic and may be substituted in any manner such that the wavelength of irradiation can be selected or so the isomerizable moiety can be attached to the crosslinker repeating unit or to a linking unit.

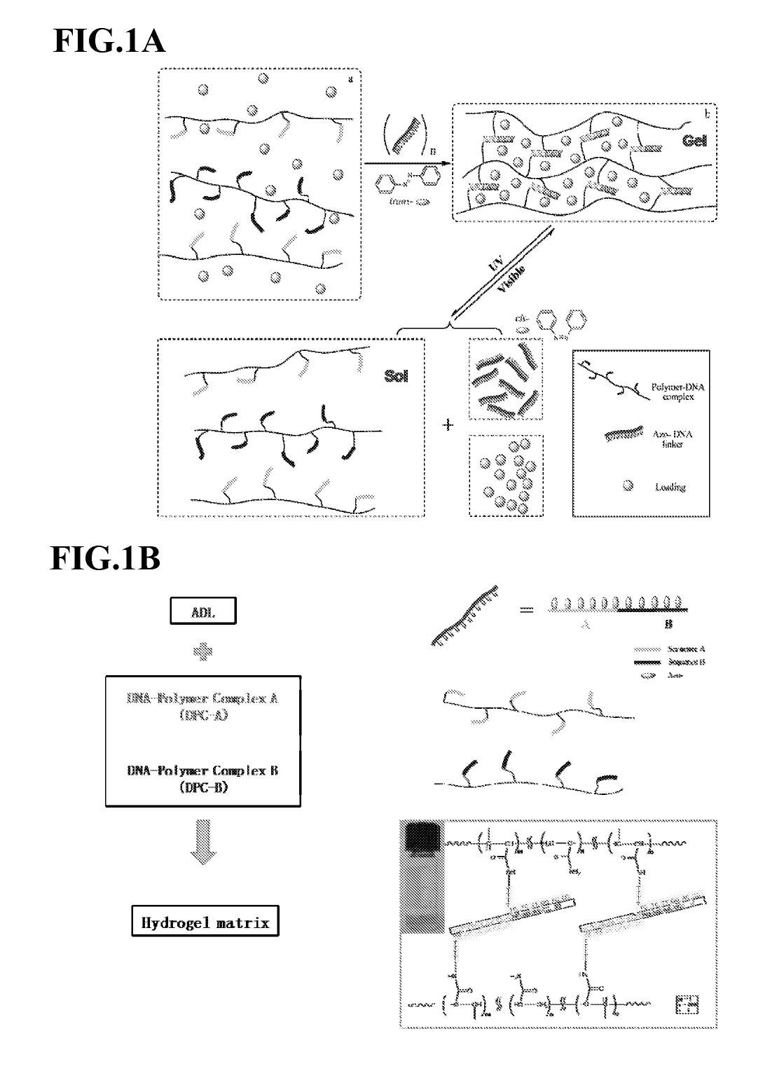

An exemplary embodiment of the hydrogel comprises linear or branched polyacrylamide chains tethered with short DNA sequences and complementary DNA linkers with isomerizable moieties to crosslink the polymer chains such that drug molecules and/or other therapeutic reagents can be incorporated into the gel state for delivery. Specifically exemplified herein is a system where an azobenzene moiety is incorporated onto one of the complementary DNA sequences' backbone such that regulation of the degree of crosslinking of the sol and gel states of the hydrogel is controlled by the photoresponsive azobenzene moieties' interconversion between cis and trans isomeric states. When the complementary DNA sequences hybridize, the polymer is in a gel state; however, when the complementary DNA sequences do not hybridize, the polymer is in a sol state. The cis and trans isomeric state of the photoresponsive moiety dictates whether the DNA sequences hybridize or S disassociate. A specific embodiment of this system is shown in FIG. 1.

In an example set forth herein utilizing 13 nm water-soluble BSA-modified gold nanoparticles as a model drug, it was demonstrated that hydrogels of the subject invention can trigger controllable release of encapsulated molecules and/or nanomaterials.

The exemplary embodiment provides many advantages, including: (a) a polyacrylamide gel that has an inert surface which minimizes nonspecific interactions, (b) all components, including polyacrylamide comprising water soluble polymers and the DNA linkers, have excellent biocompatibility and low toxicity, (c) the DNA crosslinked hydrogel system is stable, reversible and does not need special treatments for storage, transport and application, (d) the subject DNA crosslinked hydrogel system can be easily prepared in situ and can be modified as dictated to conform to the applied environment for specific dissolving requirements such that the load release from the hydrogel system can be controlled by photon energy yet allow great flexibility for different applications, such as drugs, (e) the subject hydrogel system has wide applications since the DNA crosslinking generally does not affect loaded drugs allowing the DNA crosslinking sequences to be designed for the purpose of optimizing the hydrogel delivery of its load, (f) there is no need to identify specific targets for releasing by this advantageous hydrogel delivery system since photons have a high penetration capability in vivo and are able to initiate the releasing process with localized precision with tunable controllability, and (g) pharmaceuticals or other chemicals can be efficiently encapsulated inside the DNA crosslinked hydrogel for transporting and release upon application of light stimuli. The releasing step can be interrupted whenever the input light energy is turned off. Thus, trapping, releasing and intermediate interfering of hydrogel conformation are highly controllable.

According to this embodiment of the invention, the terms azobenzene and azo are interchangeable and refer to a wide class of molecules that share the core azobenzene structure. The core azobenzene structure is composed of two phenyl rings linked by a N—N double bond. Azobenzene moieties according to an embodiment of the invention reversibly isomerize between trans- and cis-form under particular wavelengths of light. In one embodiment of the invention, azobenzene isomerizes between trans- and cis-form under UV and visible light irradiation.

The novel and advantageous DNA crosslinked polymer hydrogels can be regulated by photons. In one embodiment, gel and sol state of a hydrogel network is controlled by azobenzene moieties integrated on the DNA strand which serves as a photoresponse crosslinker. Because of the reversible association/dissociation phenomenon between azobenzene DNA linker and DNA-polymer conjugates under visible/UV light, the hydrogel system of the subject invention is engineered into convenient carriers for controlled release.

Following are examples that illustrate procedures for practicing the invention. These examples should not be construed as limiting. All percentages are by weight and all solvent mixture proportions are by volume unless otherwise noted.

### Materials and Methods

### Synthesis of Azobenzene Phosphoramidite (Azo-)

The synthesis of Azo- followed the protocol from Asanuma et al. with minor modification (Asanuma et al., 2007, Nature Protocols, 1, 203-212). The azobenzene phosphoramidite was obtained as an orange-red solid. ¹H NMR (CDCl₃): δ 8.00-6.79 (m, 22H), δ 6.62 (d, 1H), δ 4.48 (m, 1H), δ 4.39 (m, 1H), δ 4.21-4.10 (m, 2H), δ 3.77 (s, 6H), δ 3.57-3.34 (m, 4H), δ 2.76-2.72 (m, 2H), δ 1.30-1.25 (m, 15H). ³¹P (CDCL₃): δ 149.

### Synthesis of Acrydite Phosphoramidite

The compound was synthesized by two steps: first, 6-amino-1-hexanol (9.32 g, 0.08 mol) and TEA (also known as 2-(bis(2-hydroxyethyl)amino)ethanol) (16.16 g, 0.16 mol) in 100 mL dichloromethane was cooled to 0° C. Methacryloyl chloride (10 g, 0.0957 mol) was then added slowly, stirred at 0° C. for 2 hour, and 100 mL water was added to quench the reaction. The organic layer was washed with 5% HCl and dried. After evaporating all the solvent, the crude 6-hydroxyhexyl methacrylamide was used for the next step without further purification. To a solution containing 6-hydroxyhexyl methacrylamide (2 g, 10.8 mmol) in anhydrous CH₃CN (40 mL) at 0° C., N,N′ Diisopropylethylamine (D1PEA) (3.9 g, 30.0 mmol) was added dropwise over a 15 minute period. Then, 2-cyanoethyl diisopropyl chlorophosphoramidite (2.9 ml, 13 mmol) was added dropwise, and the reaction mixture was stirred at 0° C. for 5 h. After removing the solvent, the residue was dissolved in ethyl acetate, and the organic phase was washed with NaHCO₃ standard solution and NaCl solution and dried over anhydrous magnesium sulfate. Finally, the solvent was evaporated, and the residue purified by column chromatography (ethyl acetate/hexane/triethylamine 40:60:3) and dried to yield the target compound (3.33 g, 8.64 mmol, 80%) as a colorless oil. ¹H NMR (CDCl₃): δ 5.92 (br, 1H), 5.63 (m, 1H), 5.27 (m. 1H), 3.86-3.72 (m, 2H), 3.66-3.49 (m, 4H), 3.30-3.23 (m, 2H), 2.61 (t, 2H), 1.92 (m, 3H), 1.58-1.50 (m, 4H) 1.37-1.32 (m, 4H) 1.17-1.13 (m, 12H). ¹³C NMR (CDCl₃): δ 168.6, 140.4, 119.3, 118.0, 63.8, 63.6, 58.6, 58.3, 43.2. 43.1, 39.8, 31.3, 29.7, 26.8, 25.8, 24.9, 24.8, 24.7, 19.0. ³¹P (CDCl₃): δ 148.

### Synthesis of Azobenzene DNA Linker (ADL) and DNA Linker (DL)

ADL was synthesized by using a DNA/RNA synthesizer AB13400 (Applied Biosystems). A solid-phase synthesis method was used to couple FAM to the MBs' 5′ ends. The synthesis started with a 3′-Dabcyl controlled-pore glass (CPG) column at 1 μmol scale. A routine coupling program was used to couple the normal bases from 3′ end on Dabcyl CPG. After synthesis, the DNAs were cut and eluted from silica beads and chemically treated before being transferred to HPLC for purification. The final sample was dried and stored at −20° C. for future use. The DL was prepared by the same method as that used for ADL by DNA/RNA synthesizer and HPLC.

### Synthesis of DNA Polymer Conjugates (DPC)

Acrydite phosphoramidite was dissolved in acetonitrile and loaded to DNA synthesizer for two types of acrydite-modified oligonucleotides (Sequence A: 5′-Acrydite-TTTTTCACAGATGAGT-3′ (SEQ ID NO:1); Sequence B: 5′-Acrydite-TTTTCCCAGGTTCTCT-3′ (SEQ ID NO:2)). The synthesized acrydite-modified oligonucleotide monomers were further purified by reverse HPLC and quantitatively characterized by absorption at 260 nm. DPC-A and -B were prepared separately at 3 mM DNA concentration. The stock solution contained: 10 mM Tris buffer (pH 8.0), 50 mM NaCl, 10 mM MgCl₂, 4% acrylamide, 1% MW Ciba IRGACURE 2959, 3 mM DNA Sequence A or B. After mixing, UV light from a portable UV lamp (350 nm) was applied 5 cm away from this mixed solution for 18 min for copolymerization. DPC-A and -B were obtained with clear yellow color solutions with slight viscosity.

In one embodiment, Azo- was dissolved in dry acetonitrile in a vial connected to the synthesizer (20 mg Azo- can make a single incorporation in the DNA at 1.0 μmol scale synthesis). The azobenzene-tethered monomer is regarded as a normal base for insertion in programming the synthesizer. A coupling program of longer reaction time was applied to couple the 5′ FAM fluorophore at the very end terminus. After the synthesis, the CPG substrate was transferred to a glass vial, and standard AMA (ammonium hydroxide:methylamine=1:1) deprotection solution was added and incubated in water bath at 50° C. for 12 hours. After centrifuge to separate the solid beads from MB in the solution, the clear supernatant was carefully collected. Then, the DNA was concentrated by ethanol precipitation. The precipitate was redissolved by tetraethylammonium acetate (TEAA) solution and delivered to reverse phase HPLC using a C18 column with a linear elution. The collected product was then vacuum dried, detritylated, and stored at −20° C.

### Hydrogel Preparation

DNA linker (ADL or DL), DPC-A and DPC-B were mixed in stoichiometric 3 mM DNA concentrations in Tris buffer (10 mM Tris (pH 8.0), 50 mM NaCl, 10 mM MgCl₂). Crosslinked hydrogels (yellow color) or DL crosslinked hydrogel (colorless) were formed immediately after mixing. All hydrogels were treated for 10 minutes of incubation at 50° C. before tests. Other concentrations of hydrogels were prepared with direct dilution with buffer solution followed with annealing and visible light irradiation.

### Light Sources

A 60 W table lamp with a 450 nm filter was selected to trigger fast cis- to trans-conversion as the visible light source. The power is strong enough to convert the cis- to trans-transition. A portable 6 W UV light source was chosen as the UV light source. The power of the UV light source had been measured by power meter with 0.197 mW (+0.3) at the irradiated sample position.

### Cage Size Estimation of DNA Crosslinked Hydrogels

The design of all components and the crosslinking rate between two polymer backbones are the main factors in deciding the cage size (also referred to herein as hydrogel pore size) and encapsulation-release capability. Based on the ratio of each component when the hydrogel was prepared, the calculation of cage size is the sum of two neighbor DNA branches from polymers and the total length of the duplex composed of three strands. According to the ratio of acrydite-modified DNA and acrylamide (1:200), there are 200 acrylamide-repeated units between two branched DNA. From molecular modeling of a 200-repeated acrylamide chain in a straight line, the distance is approximately 49.25 nm, which stands for the size of one cage (or pore) along the polymer chain. Using a similar method to estimate chemical bond length and regarding one nucleotide unit as 3.3 nm, the length of the linkage can be calculated at 7.33 nm, which stands for the distance between two neighbor polymer chains. Therefore, the perimeter of a 2-D pore is about 113.16 nm. Considering the softness of each segment of this pore, the pore should transform with minimum inner stress, resulting in a circular pocket with a diameter of 36.02 nm.

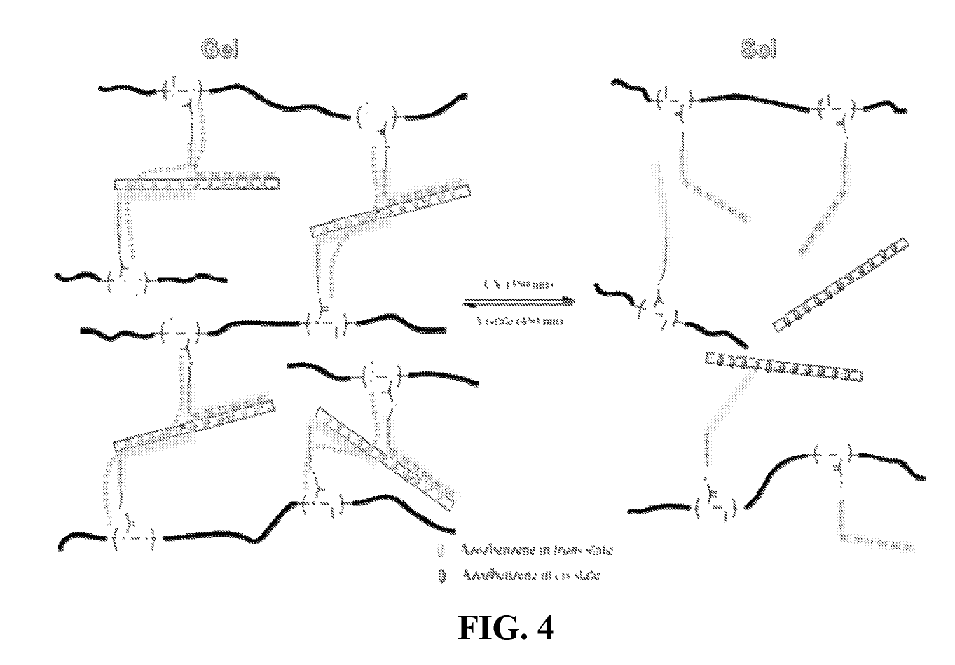

This size of cage in the subject DNA crosslinked hydrogels, especially in extreme conditions, such as >3 mM or <50 μM, will largely deviate from this value by irregular crosslinking between proximate complementary DNA strands. At high concentration, such as 3 mM, the calculated cage size (10.1 nm) is much smaller than theoretical values (36.2 nm), which means a high level of irregular crosslinking. Even at ideal concentrations, i.e., 50 μM (31.65), this irregularity will exist and generally decrease the size (FIG. 4). Consequently, it could also induce deviation of the sol-gel conversion when compared with theoretical values. Irregular crosslinking between proximate complementary DNA strands can be addressed with known methods to the skilled artisan and would decrease any likelihood of deviation of sol-gel conversion.

### EXAMPLE 1

### Design of Photocontrollable DNA Crosslinked Hydrogel

DNA linker (ADL or DL), DPC-A and DPC-B were mixed in stoichiometric 3 mM DNA concentrations in Tris buffer (10 mM Tris (pH 8.0), 50 mM NaCl, 10 mM MgCl₂). Crosslinked hydrogels (yellow color) or DL crosslinked hydrogel (colorless) were formed immediately after mixing. All hydrogels were treated for 10 minutes of incubation at 50° C. before tests. Other concentrations of hydrogels were prepared with direct dilution with buffer solution followed with annealing and visible light irradiation. UV light (˜350 nm) can photoisomerize the azobenzene moieties to cis-state, while visible light (˜450 nm) can switch the conformation back to the trans-state. The isomerization of Azo- is capable of regulating the hybridization between two complementary strands, whereby the trans-state can stabilize the hybridization, and the cis-state can destabilize it. An Azo-incorporated DNA strand was designed which serves as a photoresponse crosslinker (ADL). The ADL can crosslink two other complementary DNA strands, each one branched from comb-shaped DNA-polymer conjugates to form a hydrogel network. Because of the reversible association/dissociation phenomenon between ADL and DNA-polymer conjugates under visible/UV light, the ADL crosslinked hydrogel is expected to undergo trans-formation between gel and sol states such that it can be engineered into convenient carriers for controlled release (FIG. 1a).

Two 12-base DNA polyacrylamide conjugates (DPC-A, DPC-B) were synthesized individually by photo-initiated polymerization of 5′ acrydite-modified oligonucleotide monomer mixed with acrylamide (4%, w/v); the molar ratio of two repeating units is 1:200, respectively. ADL was designed to be 24 bases long with 11 Azo-insertions. The two half segments from ADL are complementary to each of the 12 base DNA pieces from DPC-A and DPC-B, and the 12-base DNA strands are linked to the polyacrylamide backbone by a four-T-base spacer. In order to obtain maximum sensitivity, the highest amount of Azo-moiety was inserted to the ADL to maximize photoregulation efficiency. The synthesized and purified ADL is water soluble and displays a yellow color in buffer solution. In the presence of the ADL linker, the crosslinking of two polymer chains between ADL and complementary strands from DPC-A and DPC-B immediately takes place and yields a yellow-colored hydrogel (FIG. 1b). A pretreatment of annealing (10 minutes in a 50° C. water bath) and visible light irradiation (5 minutes, 450 nm) was always performed to maximize the crosslinking and prevent possible incomplete hybridization that results from the cis-state Azo-.

The hybridization between complementary DNA strands has melting behavior as temperature increases. At temperatures higher than melting temperature (Tₘ) of DNA duplex, the duplex structure will dissociate into single-strand DNA. At temperatures lower than Tₘ, the hybridization is stable, and duplex structure is maintained. Based on the design described herein, the calculated melting temperature of the 24 base duplex strand is around 48° C., and the experimental value is 47.5° C.

### EXAMPLE 2

### Photocontrollable Sol-Gel Conversion and Reversibility

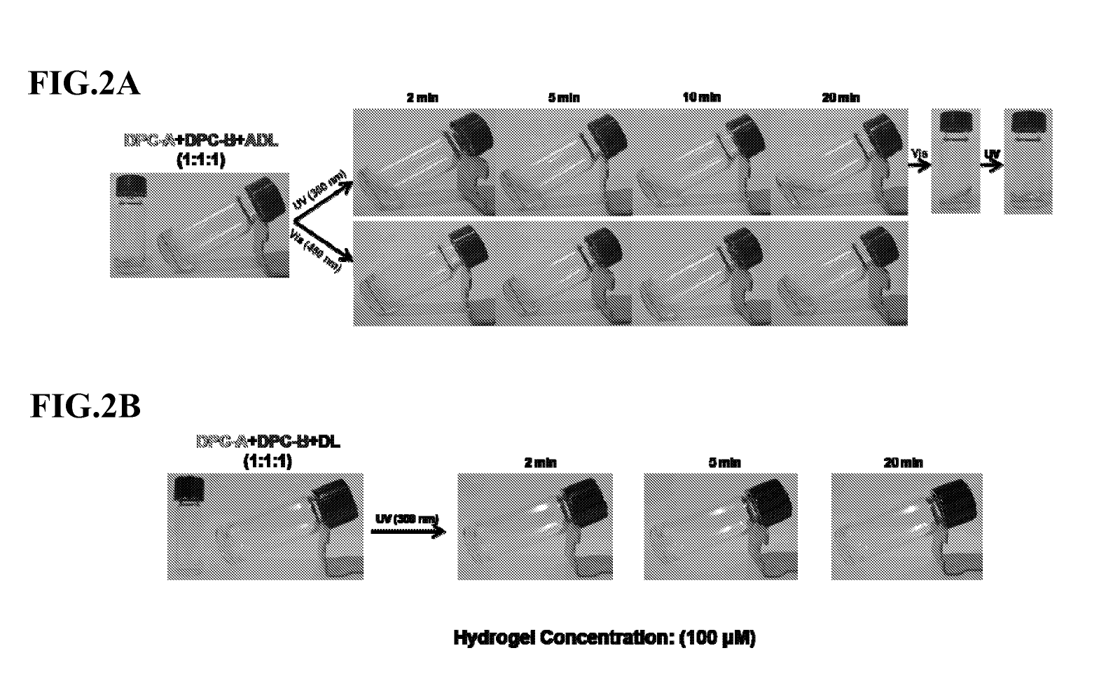

The gel-sol conversion and its reversibility were investigated through repeated UV and visible light irradiations. The prototype ADL crosslinked hydrogel was prepared by directly mixing ADL, DPC-A and DPC-B in stoichiometric concentration with 3 mM concentration hydrogel based on DNA quantity. At this concentration, the yellow hydrogel was observed as a robust gel with high viscosity. The stiffness could maintain the gel state without obvious gravitational effect similar to solid materials. The initially formed 3 mM hydrogel could be homogeneously diluted to other concentrations by annealing at 50° C. with buffer solution. These were used to investigate concentration-dependent properties, such as photo-sensitivity, efficiency of encapsulation and release. The reversible photoconversion was demonstrated with a 300 μM ADL hydrogel from dilution. This hydrogel was first irradiated by visible light and then treated with either UV or visible light. A portable UV lamp (350 nm) was used for the UV light source, and a 60 W table lamp with a 450 nm filter was used as the visible light source. The 350 nm UV light irradiation induced a melting behavior of hydrogel after 2 minutes (FIG. 2a, top row). The irradiation process lasted for approximately 20 minutes before the gel completely dissolved. The melted gel could be rapidly re-gelled with 450 nm visible light irradiation within 2 minutes. This gel-sol conversion could be repeated at least ten times without noticeable loss of conversion rate (data not shown). By contrast, the same hydrogel did not display such a melting progress under continuous visible light irradiation. However, a slight tilting of the gel was observed after approximately 20 minutes. This phenomenon may be caused by hydrogel fluidity under gravity, which was further confirmed by the subsequently performed control experiment (FIG. 2a, bottom row).

The control experiment was carried out on a 300 μM hydrogel crosslinked with plain DNA linker (DL) (FIG. 2b). The DL has a sequence identical to ADL, except no Azo-moiety. The DL crosslinked the polymer chains, DPC-A and DPC-B, in the same manner as the ADL hydrogels did. The UV irradiation up to twenty minutes only induced a slight tilting. As noted above, the tilting is very likely to be the result of gravity effect. The inert response to UV light of this plain DNA crosslinked hydrogel validates the effect of Azo-moiety, which is the key mechanism underlying the photocontrollability and reversibility of the hydrogel. Aside from the effect of gravity, ADL- and DL-constructed hydrogels might also absorb a small amount of visible light energy and thereby induce gel deformation. Both phenomena can aid a slow melting of hydrogels and seem insignificant comparing to the case of applying photon energy.

By its very nature, the viscosity of hydrogel will decrease with lower density of crosslinked scaffold. Previous synthesized DNA crosslinked hydrogels have generally required a DNA concentration above 1 mM to maintain a well-defined gel state and biofunctions. As demonstrated with the subject invention, both sol and gel states could observed at a much lower concentration of 300 μM. Besides, the hydrogel of the subject invention could be diluted down to 50 μM, while keeping enough rigidity to maintain a gel-like morphology (a hydrogel that flowed from top to bottom in a 1 mL upside down microtube took more than 1 minute; data not shown). However, as a result of the loose crosslinked hydrogel network, low-concentration hydrogels tend to deform much faster and easier than high-concentration gels, which will damage the encapsulating capability. On the other hand, high-concentration hydrogels might take longer for gel-sol conversion and thus reduce the release rate. As a consequence, there is a need to balance the gel and sol states equally so that the gel rigidity and conversion are both optimized in order to further develop their photocontrollable carrying function.

### EXAMPLE 3

### Photocontrollable Encapsulation and Release of Diverse Loads by Hydrogels

To demonstrate the photon-triggered release of loads from the photosensitive ADL crosslinked hydrogels, a series of ADL crosslinked hydrogels with different concentrations were prepared to explore the controllability. Only the factor of hydrogel concentration relative to encapsulation and release of different loads was focused, with other conditions unchanged. The entire initial doping process can be achieved by simply mixing the loads with sol-state hydrogels by either heating or UV light irradiation under stirring, followed by cooling and visible light irradiation for loading in the gel.

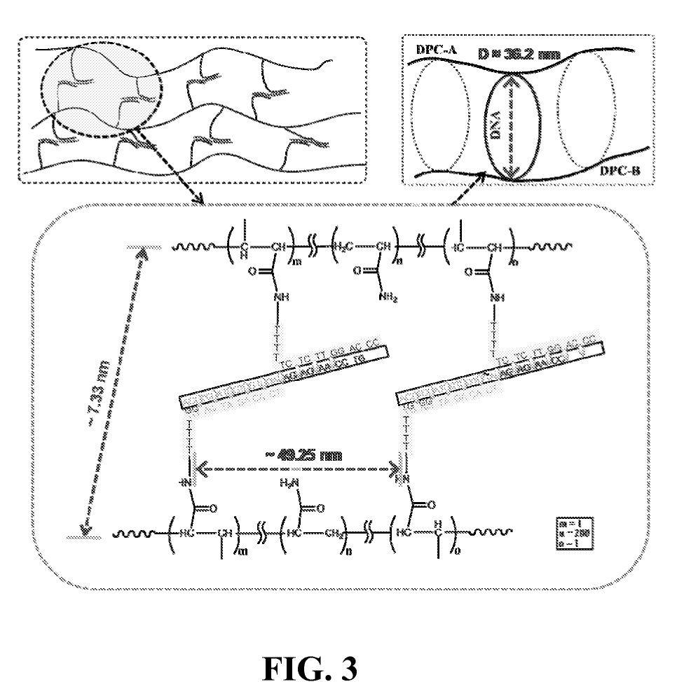

The encapsulation capability based on stability and immobility is related to the hydrogel matrix. To determine individual particle entrapment by physical size and interaction, the term “cage” was defined to represent the hydrogel network pore size. The size of the cage is estimated by the chain lengths on a regulated 2-D hydrogel structure. Two neighboring DPC polymer chains are assumed to extend on one plane and along the same direction, crosslinked by an intermediate ADL strand (FIG. 3). By modeling the chemical bond length of the cage structure, the calculated cage size along the polymer chain between two neighbor DNA branches is 49.25 nm, and the distance between two parallel neighbor polymer chains is 7.33 nm. Because of the softness of these linkages, the hydrogel was expected to have a circular structure of 36.02 nm in diameter with maximum containing capability and minimum interior stress. The isometric mixing of 3 mM concentration ensures a compact crosslinking, and the hydrogel has the average cage size of approximately 10.1 nm in diameter, as a spherical structure. It is high density packing, however, that illustrates the low crosslinking rate of most hydrogels in order to maintain the gel rigidity. 300, 100 and 50 μM hydrogels have the size of 17.42, 25.12 and 31.65 nm, respectively. Lower concentration of hydrogel by direct dilution can still keep the regularity by first increasing regular crosslinking and then decreasing it. Although pre-treatment and concentration adjustment have been applied to the hydrogels, irregular crosslinking among multiple polymer chains is inevitable on all branched polymer materials (FIG. 4).

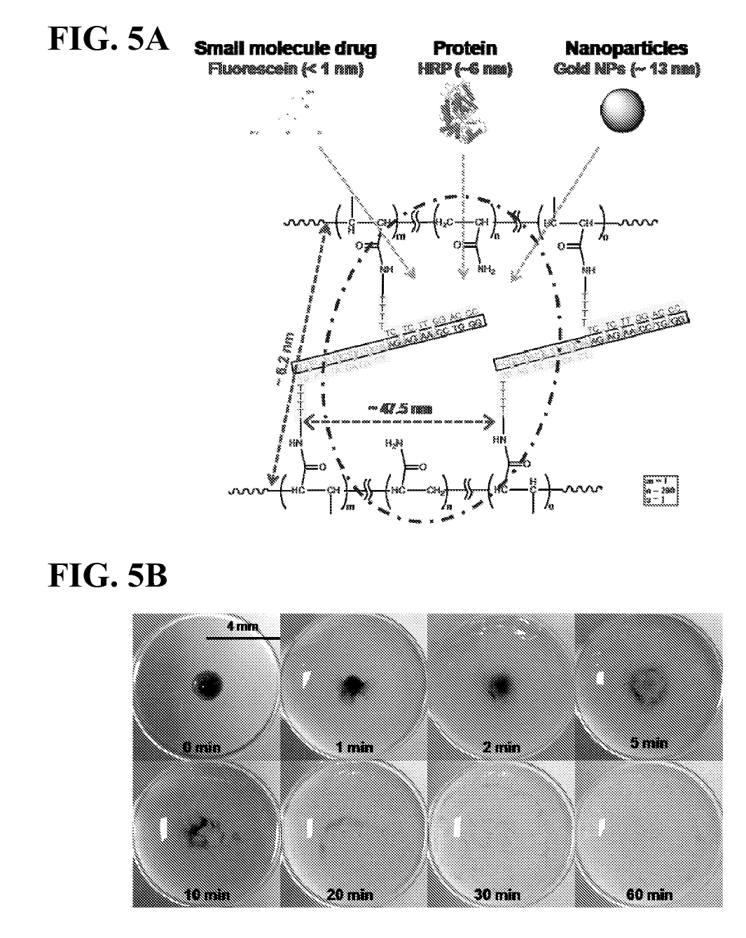

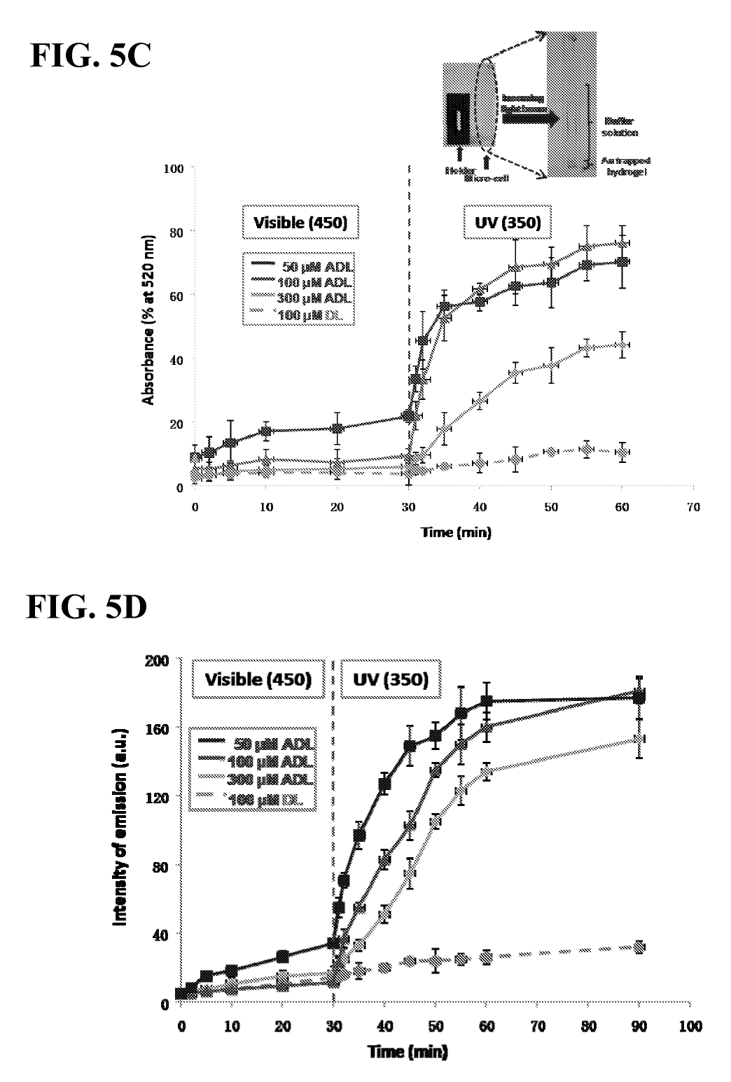

In order to study the capability and efficiency of hydrogel for controlled release, three concentration hydrogels (300, 100, 50 μM) were prepared and pre-loaded with the following: small molecule fluorescein (<1 nm), bioactive Horseradish Peroxidase (HRP) enzyme (≈6 nm), and gold nanoparticles (NPs) (13 nm) (FIG. 5a), representing a diverse set of pharmaceutical candidates: small molecule drugs, chemo- and phototherapeutic reagents and protein-based nanomedicine, respectively.

Encapsulation of loads was the same for fluorescein and gold NPs. Both materials were mixed with hydrogel by incubation at 50° C. for 10 minutes. The FIRP enzyme was incubated at 40° C. in order to keep the bioactivity. The release of fluorescein from hydrogels was monitored by direct observation and imaging by CANON SD870 digital camera. To accomplish this, 200 μM fluorescein was homogeneously dissolved in 300 μM hydrogel. 5 μL of gel mixture was dropped on a transparent plastic plate with an additional 100 μL of blank buffer. Visible and UV light were applied on top of the small reservoir, respectively.

As noted above, three different concentration ADL hydrogels, 300 μM, 100 μM, 50 μM, and one 100 μM DL hydrogel for encapsulation and releasing of gold NPs was investigated. The doping processes for all gold NPs were performed by annealing procedure with a final temperature of 20° C. A visible light irradiation was first applied for 10 minutes before increasing the temperature to prevent any interference from cis-Azo-. Then, the temperature was increased by 2° C./min until 90° C. The absorption at 520 nm was measured at every 10 degrees during the whole period. The apparent inflection points for these curves are in the range of 46-53° C., very close to the melting temperature of 47.5° C., which is the experimental melting temperature for pure DNA duplex. Most of the release processes reached plateaus at 70° C. where hydrogels were dissolved and gold NPs released to solution. In this case, the releasing process only results from melting by temperature-induced gel-sol conversion. Interestingly, 100 μM ADL and DL crosslinked hydrogels have very similar melting profiles and melting temperatures (46.9° C. and 47.5° C., respectively). All ADL crosslinked hydrogels seem to have slightly less delivery efficiency than the DL crosslinked hydrogel. We believe that this mainly results from the influence of Azo-modification to DNA linker. It was suggested in a previous study that trans-conformation Azo- can stabilize duplex structure in some cases so that the general melting process on temperature is less effective for azobenzene-modified DNA.

The release of gold NPs was monitored by absorption spectroscopy. The instrumentation included a Cary Bio-300UV spectrometer (Varian) and a pair of micro-square quartz cells (Starna Cells, Inc.). The release curves of gold NPs were obtained by calculating the absorption at 520 nm. On both light-driven and thermal-driven release, 20 μL of hydrogels were placed on the bottom of the quartz cuvette with 10 minutes of sitting time. 80 μL Tris buffer was added on top, followed by 5 to 10 minutes of visible light irradiation by a 60 W lamp and 450 nm optical filters (Asahi Technoglass). Then either light source or water bath was applied to the loaded cells, and they were immediately transferred to fluorospectrometer (Fluorolog-Tau-3, Jobin Yvon, Inc.) for absorption measurement.

The release of HRP utilized the same set of microcells. In this case, the hydrogel was placed on the bottom of a small vial, and buffer was added on the top. After each light irradiation, 1 μL supernatant was transferred to a 2 mL vial and mixed with luminal and hydrogen peroxide in buffer and stirred for 10 minutes. The emission curves were obtained by calculating the chemiluminescence at 410 nm after 10 minutes. Since the luminescence intensity is in proportion to the HRP concentration, the released HRP amount can be calculated by chemiluminescence intensity emitted from oxidation reaction.

The small-sized fluorescein molecule is a commonly used material for labeling and tracking because of its observable bright orange color and detectable strong fluorescence. More importantly, the size and physical properties of fluorescein are very similar to many chemotherapy drugs. For the fluorescein loaded hydrogel, there was no observable color diffusion from gel to surrounding buffer solution after more than 30 minutes of visible light irradiation at room temperature (25° C.). After applying UV light, however, the hydrogel mixture started to melt, and fluorescein molecules were observed to rapidly diffuse out to buffer solution (FIG. 5b). This dissolving was not caused by a strong absorption of UV light, proved by a control experiment in which fluorescein dissolved with DL crosslinked hydrogel had no response to either visible or UV light. It is interesting to observe the stable encapsulation and induced release since the fluorescein molecule is much smaller than the calculated cage size. It is believed that one main factor underlying this phenomenon is the inert mobility of fluorescein molecules in the large hydrogel pocket without an external driving force for self-diffusion, which significantly prolongs the retention time of the trapped molecules. Two other hydrogels with concentrations of 100 and 50 μM were tested under the same conditions, and the diffusion processes seemed faster with uncontrollable leaking due to larger hydrogel cage (data not shown).

13 nm water-soluble BSA-modified gold NPs were selected to further study the entrapment/releasing capability of the subject hydrogels. In order to specifically study the relationship between hydrogel concentration and doped loads, three different hydrogel concentrations, 300 μM, 100 μM and 50 μM, were prepared and encapsulated each with 500 nM gold NPs. A small portion of gel mixture was placed on the bottom of a quartz microcell (FIG. 5c, insertion) with buffer solution on top. When irradiated with UV light, the hydrogel started to melt, and the trapped gold NPs were released to the top buffer solution and quantitatively monitored by strong gold NP absorption at 520 nm at each interval (FIG. 3c). The hydrogels were initially irradiated with thirty minutes of visible light to evaluate the leaking effect before applying UV light.

The absorption curves show that the UV light dissolved the hydrogels rapidly after 1 minute, and gold NPs were released to buffer solution. Both 300 μM and 100 μM hydrogels can steadily encapsulate NPs without leaking, while the 50 μM hydrogel seems unable to enclose the particles tightly. The UV light dissolved the hydrogels rapidly after 1 minute, and gold NPs were released to buffer solution. The absorption curves also demonstrate different release rates of gold NPs under UV light irradiation on different gel concentrations.

All hydrogels seem to have an initial bursting release period and then slowing down. For the 300 μM hydrogel, the release rate was comparably slow and lasted for more than 15 minutes before reaching a plateau with an average rate of 1.96±0.19×10⁻⁴ nmole/min. The 100 μM hydrogel seemed to have a higher rate of release upon UV irradiation. The average release rate was 3.95±0.36×10⁻⁴ nmole/min before reaching a plateau at 15 minutes. Despite a serious problem in uncontrollable leaking, the evenly diluted 50 μM hydrogel is the fastest to reach the plateau with a release rate of 6.88±0.70×10⁻⁴ nmole/min in 5 minutes. This difference among the three hydrogels clearly demonstrated concentration-dependent encapsulating and releasing capability under light irradiation. It seems that the 100 μM hydrogel has the best balance overall with stable containment effect and rapid release rate, properties which actually correlate well with the stability of the hydrogels' gel and sol states. Besides the release rate, the net amount of gold NPs released from each concentrated hydrogel is also an important factor in evaluating the delivery capacity. The 300 μM hydrogel displays high resistance in releasing gold NPs by UV illumination, and only 38.1% of NPs were released after 30 minutes of visible and UV light, while the 100 and 50 μM hydrogels could release up to 66.9% and 48.4% during the same period. A 100 μM DL crosslinked hydrogel displays weak response to both visible and UV light on both releasing rate and amount (less than 10% overall releasing). This concentration-dependent property can be explained by the cage size composed of DPC backbone and ADL linker on different DNA concentrations. Although the size of gold NPs is smaller than that of the theoretically calculated cage size for an ideally crosslinked hydrogel, the interaction between cage skeleton and gold NPs is able to balance the retention and diffusion rates.

The small molecule fluorescein dye and large size gold NPs are generally structurally stable and will not change activity in most transporting methods or materials. However, for bioactive enzymes and macromolecules, invasive carriers, which need chemical binding or strong physical adsorption, might have their functional structure irreversibly altered with resulting damage to their bioactivity. Therefore, in this experiment, the hydrogels were used to deliver HRP as a bioactive drug model. HRP enzyme has both bioactivity and specificity on specific substrates and is widely used as a preferred enzymatic label (Dick et al., 1999, Cardiovasc Intervent Radiol, 22, 389-393). The visible existence of HRP enzyme can be demonstrated by quantitative chemiluminescence from substrate oxidization. Similar to the visible/UV irradiation applied to gold NPs, the HRP-loaded hydrogels were examined with kinetic release, and the luminescence profiles were recorded by spectrophotometer, which were further converted to HRP amount released to buffer solution (FIG. 5d). All three ADL crosslinked hydrogels with different density have typical UV switch-on releasing profiles. The 300 μM hydrogel had a better performance in releasing HRP in this case, and diluted 50 μM hydrogel had the fastest release rate. The calculated results demonstrated a net releasing of 46.7%, 59.2% and 56.0% of active HRP after 60 minutes of UV irradiation released from 300 μM, 100 μM and 50 μM ADL hydrogels, and 4.8% for the control 100 μM DL hydrogel, respectively. These results strongly demonstrated a successful delivery of a bioactive protein by our photocontrollable hydrogels. The activity of the enzyme molecules was confirmed by the enzymatic reaction. Comparably, the 100 μM hydrogel still has the best balance of storing enzymes under visible light and rapidly releasing them under UV light. Without azobenzene moiety, the 100 μM DL crosslinked hydrogel could only store the enzymes with a slight response to the light irradiations.

### EXAMPLE 4

### Thermodynamic Response of the Hydrogels and Their Biocompatibility

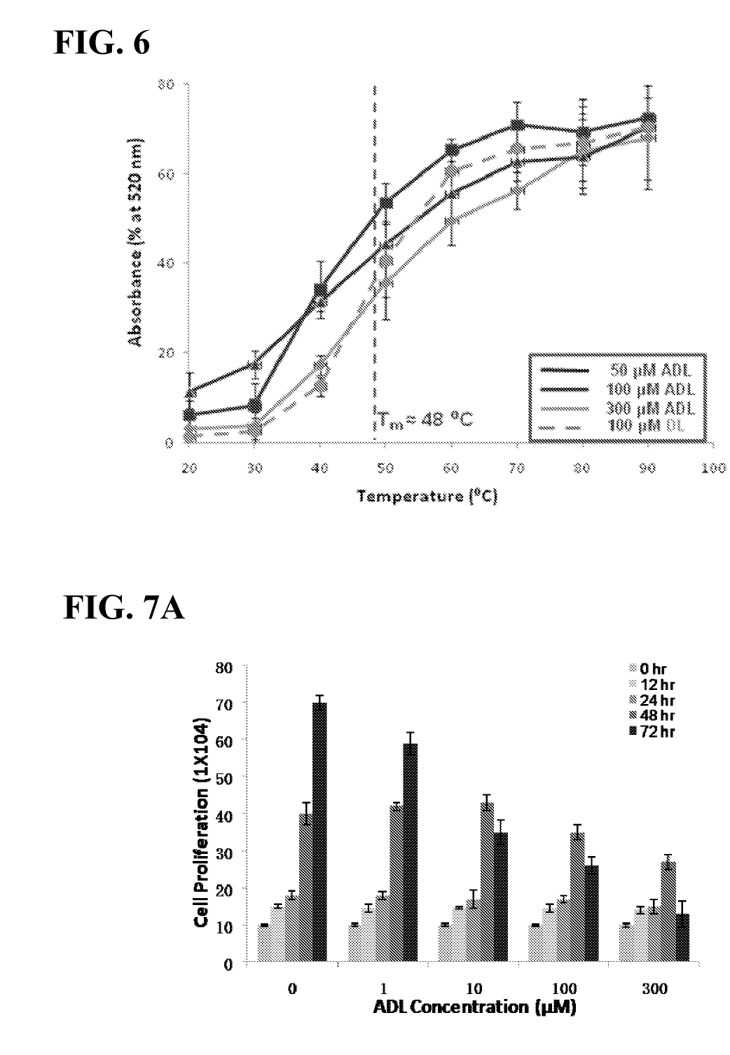

Besides the photocontrollability brought by photosensitive Azo-moiety for multiple-load releasing, the DNA crosslinked hydrogels might have a similar reversible thermodynamic melting property independent from light energy. The melting property can also be regarded as a gel-sol conversion independent of Azo-moiety and external light energy and defined only by DNA sequences. Therefore, three ADL hydrogels, 300 μM, 100 μM, 50 μM, and one 100 μM DL hydrogel, were investigated for encapsulation and releasing of gold NPs (FIG. 6). As expected, all DNA crosslinked polymer hydrogels had melting profiles very similar to that of the pure DNA duplex without polymer backbone. The results of thermodynamic releasing are very consistent with the photocontrol of gold NPs with hydrogel concentration dependency. Since this thermo-response property of ADL crosslinked hydrogel is independent of Azo-modification, it provides an additional factor in controlling the release of loads.

Biocompatibility of the hydrogels was also investigated by incubating hydrogel samples with cells. ADL and ADL hydrogels were prepared with different concentrations and mixed with the same amount of CEM cells (1 million/ml). The cytotoxicity of each sample was calculated by counting cell proliferation at 0, 12, 24, 48 and 72 hours. The proliferation was obtained by counting living cells under the microscope. The distribution of cells at different stages was monitored by Vybrant Apoptosis Assay Kit #2 (Invitrogen) and flow cytometry (FACScan cytometer, Becton Dickinson Immunocytometry Systems).

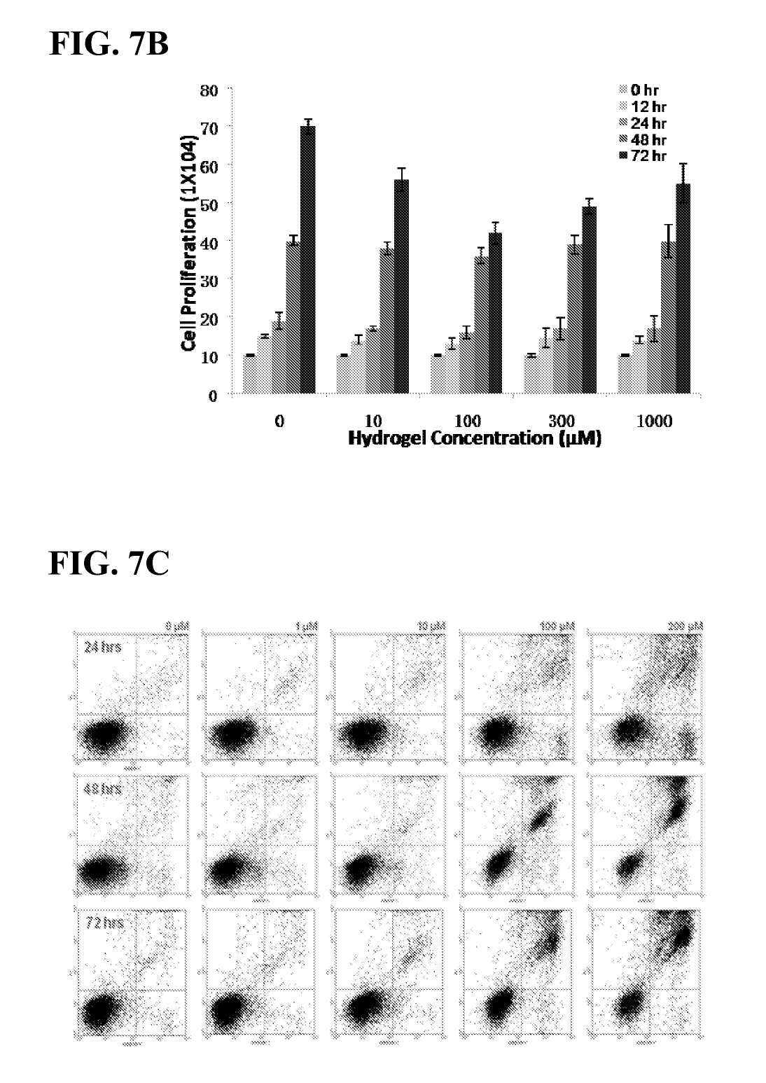

Different concentrations of DNA linkers and hydrogels were mixed with human leukemia CEM cells, and cell proliferation was monitored by counting living cells and monitoring cell apoptosis at different stages from 12 to 72 hours for ADL (FIG. 7). CEM cell samples incubated with ADL or ADL hydrogels were treated with apoptosis reagent, Alex Fluor 488 annexin V/propidium iodide (Vybrant Apoptosis Assay Kit #2, Invitrogen) and applied with flow cytometry.

### Biocompatibility of ADL

Leukemia CEM cells were incubated at 37° C. in cell culture medium. Cells from a simple bottle were aliquoted and used for each batch of experiments. The number of cells was maintained at 100, 000 for each well for cytotoxicity investigation. When mixed with DNA linkers and hydrogels, apoptosis reagent was added to cell medium at the same time to label dying and unhealthy cells. The cell proliferation-based cytotoxicity was monitored by flow cytometry from 12 to 72 hours (Supplementary FIG. 7a). The toxicity of ADL to CEM cells modeled final conditions when the ADL hydrogel totally dissolved and all ADL were released to cell culture solution. Only at concentrations higher than 100 μM and incubation of more than 48 hours do the results show that a significant toxicity was observed. This indicates that the Azo-DNA linker is safe to be used for this cell line under the concentration of 100 μM.

### Biocompatibility of ADL Hydrogels

Four different concentrations of ADL crosslinked hydrogels were prepared for cytotoxicity to CEM cells. Each concentration gel was mixed with identical cell solution, and the cell proliferations were monitored on a timeline (Supplementary FIG. 7b). The calculated final concentrations of ADL and DNA-polymer conjugates are 1, 10, 30 and 100 μM, respectively. The result displays a comparable low toxicity for low- and high-concentration hydrogels. At the concentration of 10 μM, the hydrogel is very dilute in cell medium, and the crosslinking ratio is very low. As a result, the pore size of this gel is large enough to permit cells to go inside the gel matrix and freely make contact with DPC and ADL. For this reason, it has a slightly lower toxicity than the 1 μM ADL in cell medium, while the DPC should contribute the rest of toxic effect. For high-ratio crosslinked ADL hydrogels, such as 1000 μM, the highly crosslinked polymer network can maintain a compact morphology and prevent the cells from entering the hydrogel. Therefore, the effective concentrations of ADL from these hydrogels are much less than those of ADL. It is only when the gel is totally dissolved that a large amount of free ADL could be more toxic. The middle concentrations of gels (100 and 300 μL) have a higher cytotoxicity caused by comparably looser structures and higher toxicity than 1000 μM gel. These results indicate that the Azo-DNA crosslinked hydrogels do not affect cell growth under the experimental conditions described above.

As described herein, the benefits of the DNA crosslinked hydrogels of the subject invention are many. They include: inexpensive production (all components needed to prepare the subject hydrogels, including polymer and DNA sequences, can be obtained easily and economically), easy of handling (the subject hydrogel is stable in ambient condition, and the preparing, storing, shipping and applying for treatment are easy to perform), in situ preparation with therapeutics (the hydrogel system can be easily prepared in situ and mixed with many pharmaceuticals), high efficiency (the subject hydrogel system can encapsulate a large amount of pharmaceuticals and chemicals and has high efficiency for localized high dosage release), highly tunable (the crosslinking to form the hydrogel is solely controlled by DNA hybridization so that the hydrogel morphology can be finely engineered by sequence designing as well as polymer modification), controllable load releasing (the releasing process is controlled by photon energy, which can trigger a burst releasing comparing to a layering response in most hydrogel delivery systems; moreover, the releasing can be initiated and paused at any interval. Further, the releasing can be further controlled on a selective region by precise manipulation of light irradiating extent), high bio-safety (the subject hydrogel system is composed of non-toxic materials with high biocompatibility).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A photocontrollable hydrogel comprising:
(a) a water soluble polymer comprising at least two repeating units;
(b) at least one crosslinker; and
(c) at least one photoresponsive isomerizable group.

2. The photocontrollable hydrogel of claim 1, wherein the water soluble polymer comprises a homopolymer, a copolymer of two or more water soluble repeating units, or a copolymer of one or more repeating unit of a water soluble homopolymer and one or more repeating unit of a water insoluble homopolymer such that the resultant copolymer is water soluble.

3. The photocontrollable hydrogel of claim 1, wherein the water soluble polymer comprises linear, branched, hyperbranched or dendritic units.

4. The photocontrollable hydrogel of claim 1, wherein the repeating unit is polyacrylamide, polyvinyl alcohol, polyacrylic acid, polyethylene oxide, or modified cellulose.

5. The photocontrollable hydrogel of claim 1, wherein the photoresponsive isomerizable group is azobenzene or 1,2-diphenylethylene.

6. The photocontrollable hydrogel of claim 1, wherein the crosslinker comprises a pair of complementary nucleic acid sequences, wherein each of the nucleic acid sequences is copolymerized with, attached to, or coupled with the water soluble polymer, and wherein one of the complementary nucleic acid sequences includes the photoresponsive isomerizable group.

7. The photocontrollable hydrogel of claim 6, wherein the nucleic acid sequences are made from DNA, RNA, or a mixture of DNA and RNA.

8. The photocontrollable hydrogel of claim 1, wherein the crosslinker comprises a pair of components that are operably linked or associated, wherein each of the components is copolymerized with, attached to, or coupled with the water soluble polymer, and wherein one of the components includes the photoresponsive isomerizable group.

9. The photocontrollable hydrogel of claim 1, wherein the crosslinker comprises: (a) at least one nucleic acid sequence copolymerized with, attached to, or coupled with the water soluble polymer and (b) a linking unit comprising the photoresponsive isomerizable group and a nucleic acid sequence that is complementary to the at least one nucleic acid sequence copolymerized with, attached to, or coupled with the water soluble polymer.

10. The photocontrollable hydrogel of claim 9, wherein the nucleic acid sequences are made from DNA, RNA, or a mixture of DNA and RNA.

11. The photocontrollable hydrogel of claim 1, wherein the crosslinker comprises (a) at least one component copolymerized with, attached to, or coupled with the water soluble polymer and (b) a linking unit comprising the photoresponsive isomerizable group and a component that is complementary to the at least one component copolymerized with, attached to, or coupled with the water soluble polymer.

12. The photocontrollable hydrogel of claim 1, further comprising a therapeutic agent.

13. The photocontrollable hydrogel of claim 12, wherein the therapeutic agent is a drug.

14. A method for photocontrolling the gel and/or sol states of a hydrogel, said method comprising:
(a) providing a photocontrollable hydrogel comprising a water soluble polymer comprising at least two repeating units; at least one crosslinker; and at least one photoresponsive isomerizable group; and
(b) exposing the photocontrollable hydrogel to photons to control the gel and/or sol state of the photocontrollable hydrogel.

15. The method of claim 14, wherein the water soluble polymer comprises linear, branched, hyperbranched or dendritic units of polyacrylamide.

16. The method of claim 14, wherein the photoresponsive isomerizable group is azobenzene.

17. The method of claim 16, wherein the photocontrollable hydrogel is exposed to UV and visible light irradiation to control the gel and/or sol state of the photocontrollable hydrogel.

18. The method of claim 14, wherein the photocontrollable hydrogel further comprises a therapeutic agent and wherein control of the gel and/or sol state of the photocontrollble hydrogel also controls the release of the therapeutic agent.

19. The method of claim 14, wherein the crosslinker comprises a pair of complementary nucleic acid sequences, wherein each of the nucleic acid sequences is copolymerized with, attached to, or coupled with the water soluble polymer, and wherein one of the complementary nucleic acid sequences includes the photoresponsive isomerizable group.

20. The method of claim 14, wherein the crosslinker comprises: (a) at least one nucleic acid sequence copolymerized with, attached to, or coupled with the water soluble polymer and (b) a linking unit comprising the photoresponsive isomerizable group and a nucleic acid sequence that is complementary to the at least one nucleic acid sequence copolymerized with, attached to, or coupled with the water soluble polymer.
